(19) United States
(12) Patent Application Publication
(10) Pub. No.: US 2009/0105560 A1
(43) Pub. Date: Apr. 23, 2009

(54) LIFESTYLE AND EATING ADVISOR BASED ON PHYSIOLOGICAL AND BIOLOGICAL RHYTHM MONITORING

(76) Inventor: DAVID SOLOMON, Zihron Yaaqov (IL)

Correspondence Address: The Law Office of Michael E. Kondoudis, PC, 888 16th Street, N.W., Suite 800, Washington, DC, 20006, US

(21) Appl. No.: 12/341,945

(22) Filed: Dec. 22, 2008

Related U.S. Application Data
(63) Continuation-in-part of application No. PCT/IL2007/000782, filed on Jun. 27, 2007.

(51) Int. Cl. G06Q 90/00; A61B 5/00; G09B 19/00

(52) U.S. Cl. 600/301; 434/127; 705/1

(57) ABSTRACT

A computerized system for scheduling at least one daily activity of a user. One or more sensors are attached to the body of the user which monitor one or more physiological parameters of the body Physiological data is produced representative of the one or more physiological parameters during a time period. A processing unit attached to memory, is programmed for the scheduling of activities based on the physiological data and on previously stored values. The scheduled activities preferably include eating of a meal, exercise or rest of the user. Physiological parameters include skin temperature and/or heart rate. When the scheduled daily activity is eating of a meal, the processing unit is preferably programmed to recommend to the user to eat the meal during a portion of the time period when the skin temperature is rising or when the heart rate is falling.

### CROSS REFERENCE TO RELATED APPLICATIONS

The present application is a continuation-in-part application of PCT international application PCT/IL2007/000782, currently pending, filed on 27 Jun. 2007 by the present inventor.

### BACKGROUND

1. Technical Field

The present invention relates to a system and method which monitors physiological parameters in people, and scheduling activities based on the monitored parameters, and more particularly, to daily scheduling of meal times and exercise based on monitoring of skin temperature, physical activity and/or heart rate.

2. Description of Related Art

The concept of biological rhythm includes self-sustained and cyclic change in a physiological process or behavioral function of an organism that repeats at semi-regular intervals. A circadian rhythm is a self-sustained biological rhythm which in an organism's natural environment is normally synchronized to a twenty four hour period. Circadian rhythm of about 24 hours is related to daily activities and rest during the twenty four hour daily cycle. An infradian biological rhythm has a cycle of more than twenty four hours; for example, the human menstrual cycle. An ultradian rhythm is a biological rhythm with a cycle of less than twenty four hours such as in human sleep cycles and the release of some hormones related to physiological functions such as ingestion.

Biological rhythms may be understood based on autonomic nervous system balance between sympathetic autonomic nervous system which increases in function during physical activity and the parasympathetic nervous system which increases in function during rest. In terms of metabolism, different phases of biological rhythm are characterized by catabolic metabolism i.e. burning of glucose and anabolic metabolism, i.e., synthesis of glucose and fat.

Biological rhythm is strongly influenced by lifestyle particularly by timing of physical activity, food and rest/sleep. Physical exercise increases sympathetic autonomic nervous system function, while eating and rest induce parasympathetic autonomic nervous system function. Sedentary lifestyle increases anabolic metabolism and leads to decreased sympathetic autonomic nervous system function and affects biological rhythm accordingly.

Obesity has become a major health problem in many developed and developing countries. In the USA, about thirty percent of the population is considered to be obese and fifty percent over optimal weight and about forty-six percent of Americans are actively trying to lose weight. People in modern society suffer from eating disorders in some cases due to social and psychological factors that disturb natural digestion regulation processes. Stress and psychological factors influence eating behavior and lead to eating disorders. Physical activity is known to be favorable for weight control and particularly burning fat. Some modern diets propose eating every three hours in order to decrease sugar fluctuations and avoid overeating during the state of being too hungry.

There is extensive prior art in the area of monitoring of physiological parameters on the human body. Stivoric et al. (U.S. Pat. No. 7,153,262) discloses a sensor array and computing apparatus located on the human body while maintaining the sensors and apparatus within a proximity zone of the body such that the mobility and flexibility of the body are not deleteriously affected by the presence of the apparatus. The system permits the dynamic monitoring of human physiological status data without substantial interference in human motion and flexibility. A processor is mounted within a pod location with or adjacent to a sensor pod location, or the processor may be electrically connected to the sensor.

Teller et al. (U.S. Pat. No. 6,605,038) discloses a system for detecting, monitoring and reporting physiological information includes a sensor device adapted to be worn on the upper arm that includes at least one of an accelerometer, a GSR sensor and a heat flux sensor and generates data indicative of at least one of physical activity, galvanic skin response and heat flow. The sensor device may also generate derived data from at least a portion of the data indicative of at least one of physical activity, galvanic skin response and heat flow. The system includes a central monitoring unit that generates analytical status data from at least one of the data indicative of at least one of physical activity, galvanic skin response and heat flow, the derived data, and previously generated analytical status data, a means for establishing electronic communication between the sensor device and the central monitoring unit, and a means for transmitting data to a recipient.

The disclosures of U.S. Pat. Nos. 7,153,262 and 6,605,038 are included herein by reference for all purposes as if entirely set forth herein.

Maximal oxygen consumption, maximal oxygen uptake or aerobic capacity) VO2 max is the maximum capacity of an individual's body to transport and utilize oxygen during incremental exercise, which reflects the physical fitness of the individual. VO2 max is expressed either as an absolute rate in liters of oxygen per minute (l/min) or as a relative rate in milliliters of oxygen per kilogram of bodyweight per minute (ml/kg/min).

The term “physiological parameter” is used herein refers to any parameter related to function of the human body including but not limited by: skin temperature, heart rate, heart rate variability, other heart rhythms as measured by an electrocardiogram, heat flow from the body, galvanic skin response, blood pressure, blood glucose, skin color, body movement and/or physical activity, encephalographic and/or other neural activity.

### BRIEF SUMMARY

The term “physical activity” as used herein refers to a physiological state measurable by body movement or heart rate. The term “daily activity” as used herein refers to typically daily human activities such as eating, sleeping and physical exercise, which may be scheduled based on physiological data or measured signs, according to embodiments of the present invention.

Each person differs in lifestyle, daily activity and metabolism. Shift laborers, international travelers, emergency and security personnel typically suffer from disturbed biological rhythms because of the variations in lifestyle from day to day. Overweight individuals typically have disturbed biological rhythms. Balancing the biological rhythm has a positive effect on weight control. There is thus a need for, and it would be highly advantageous to have a system and method which schedules daily activities including eating and exercise times according to measured physiological or other parameters rather than at given a priori intervals, e.g., every three hours.

According to the present invention there is provided a computerized system for scheduling at least one daily activity of a user. One or more sensors are attached to the body of the user which monitor one or more physiological parameters of the body Physiological data is produced representative of the one or more physiological parameters during a time period. A processing unit attached to memory, is programmed for the scheduling of activities based on the physiological data and on at previously stored values. The scheduled activities preferably include eating of a meal, exercise or rest of the user. The previously stored values are preferably updated using statistical analysis of the physiological data. Physiological parameters include skin temperature and/or heart rate. The previously stored value include: maximum heart rate, minimum heart rate, maximum time derivative of heart rate, minimum time derivative of heart rate, maximum skin temperature, minimum skin temperature, maximum time derivative of skin temperature, minimum time derivative of skin temperature, change of temperature between meals and time between meals. When the scheduled daily activity is eating of a meal, the processing unit is preferably programmed to recommend to the user to eat the meal during a portion of the time period when the skin temperature is rising or when the heart rate is falling. When the scheduled daily activity is exercise, the processing unit is preferably programmed to recommend to the user to exercise after the skin temperature peaks and begins to decrease or after the heart rate reaches a minimum and begins to increase.

When the scheduled daily activity is rest, processing unit is preferably programmed to recommend to the user to rest when the skin temperature has reached a value higher than a previously stored threshold skin temperature value or when the heart rate is below a previously stored threshold heart rate value. When during said time period the skin temperature is above a previously defined threshold for longer than a previously defined time interval or the heart rate is lower than previously defined threshold for longer than a previously defined time interval the scheduling includes postponing a meal and recommending exercise to reduce the skin temperature and increase the heart rate. An input mechanism is used for entering by the user metadata pertaining to the user, the metadata includes: hunger level, energy level, meal time, and size of meal, and the scheduling is further based on the metadata. When the physiological data is indicative of a high stress level, the scheduling includes delaying a meal until the stress level is reduced. The physiological parameters are optionally heat flow, galvanic skin response, blood pressure, blood glucose, skin color, body movement and/or physical activity. Preferably, the scheduling is based on the physiological data of a single physiological parameter.

According to the present invention there is provided a method for scheduling at least one daily activity of a user, providing at least one sensor attached to the body of the user. The sensor monitors at least one physiological parameter of the body thereby producing physiological data representative of the at least one physiological parameter during a time period. The daily activity is scheduled based on the physiological data and on at least one previously stored value. The previously stored values are preferably updated using statistical analysis of the physiological data. Physiological parameters include skin temperature and/or heart rate. The previously stored value include: first and second order statistical values (mean and variance) of skin temperature and heart rate, maximum heart rate, minimum heart rate, maximum time derivative of heart rate, minimum time derivative of heart rate, maximum skin temperature, minimum skin temperature, maximum time derivative of skin temperature, minimum time derivative of skin temperature, typical change of temperature between meals and time between meals. When the scheduled daily activity is eating of a meal, the processing unit is preferably programmed to recommend to the user to eat the meal during a portion of the time period when the skin temperature is rising or when the heart rate is falling. When the scheduled daily activity is exercise, the processing unit is preferably programmed to recommend to the user to exercise after the skin temperature peaks and begins to decrease or after the heart rate reaches a minimum and begins to increase.

When the scheduled daily activity is rest, processing unit is preferably programmed to recommend to the user to rest when the skin temperature has reached a value higher than a previously stored threshold skin temperature value or when the heart rate is below a previously stored threshold heart rate value. When during said time period the skin temperature is above a previously defined threshold for longer than a previously defined time interval or the heart rate is lower than previously defined threshold for longer than a previously defined time interval the scheduling includes postponing a meal and recommending exercise to reduce the skin temperature and increase the heart rate. The user enters metadata pertaining to the user, the metadata includes: hunger level, energy level, meal time, and size of meal, and the scheduling is further based on the metadata. When the physiological data is indicative of a high stress level, the scheduling includes delaying a meal until the stress level is reduced. The physiological parameter is optionally galvanic skin response, blood pressure, blood glucose, skin color, body movement and/or physical activity. Preferably, the scheduling is based on the physiological data of a single physiological parameter.

According to the present invention there is provided a computer encoded with processing instructions for causing a processor to execute a method for collecting physiological data from a sensor attached to the body of the user. The the sensor monitors a physiological parameter of the body. Physiological data representative of the physiological parameter is produced during a time period. A daily activity is scheduled based on the physiological data and further based on a previously stored value.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

The invention is herein described, by way of example only, with reference to the accompanying drawings, wherein:

FIG. 1 is a graph of skin temperature as monitored on a subject indicated preferred meal times, according to an embodiment of the present invention

FIG. 2 is a system drawing according to an embodiment of the present invention;

FIG. 3 is a flow diagram of a process of scheduling meal times, according to another embodiment of the present invention

FIG. 4 is a graph of skin temperature showing recommended times for exercise, meals and rest, according to a features of the present invention;

FIG. 5 is a graph of data from an obese women with poor biological rhythm;

FIG. 6 is illustrates graphically a correlations between skin temperature, heart rate and body movement, according to an aspect of the present invention; and

FIG. 7 is a phenomenological drawing of a “state machine” indicating correlations between skin temperature, heart rate and body movement during normal physical activity, rest and exercise.

### DESCRIPTION OF EMBODIMENTS

Reference will now be made in detail to embodiments of the present invention, examples of which are illustrated in the accompanying drawings, wherein like reference numerals refer to the like elements throughout. The embodiments are described below to explain the present invention by referring to the figures. Different embodiments of the present invention include systems and methods for scheduling daily activities including eating and exercise according to monitored physiological parameters such as skin temperature or heart rate. During rest or sleep, body skin temperature typically rises while heart rate decreases. Skin temperature rises during rest due to a decrease of sweating and vasodilation. During physical activity the body skin temperature decreases and the heart rate increases. Skin temperature decreases during activity due to vasoconstriction and increased sweating. After a meal there is an increase of metabolism producing heat thereby which causes an increase of heart rate, and in some cases an eventual increase of skin temperature. Hunger is associated with a decrease of heart rate down to a basal heart rate when the person is inactive and a decrease of metabolic heat production or a decrease of heat flow from the body to the environment). Thus skin temperature, evaporation and heat flow are parameters which reflect metabolic changes associated with biological rhythm and activity of the body.

Before explaining embodiments of the invention in detail, it is to be understood that the invention is not limited in its application to the details of design and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

By way of introduction, principal intentions of embodiments of the present invention are to: monitor one or more physiological parameters characteristic of biological rhythm and use the monitored parameters to schedule daily activities particularly meal times, exercise and rest/sleep. Embodiments of the present invention herein are intended for personal scheduling of daily activities to achieve weight control. Other embodiments of the present invention may be used by individuals for improving efficacy of sleep and/or reducing stress. The present invention in certain embodiments may be useful to schedule ingestion or injection of medications or other treatments.

A basic concept relevant to embodiments of the present invention is that the digestion system: stomach, gallbladder, liver, etc. need to “warm-up” and be ready for receiving food and the digestion process requires secretion of hormones and digestive juices performed in the body to enable proper digestion. In a natural situation the person feels hunger which signals the body to prepare for eating food. However, people commonly fail to recognize subtle body signals or are unable to eat due to lifestyle constraints and optimal times for eating are missed. Often overeating results when the optimal meal time is missed and the persons becomes too hungry for a long period of time. Alternatively, people eat snacks frequently and eat large meals at socially convenient times even when they are not hungry. Eating when the digestive system is not prepared for digestion creates frequent digestion cycles and eventually to a chronically increased appetite and eventual obesity. In addition to the preparation of the digestive organs the healthy biological rhythm typically includes periodic variations anabolic and catabolic phases or—eat-rest—activity cycles that enable metabolizing the ingested food and preparing for the next daily activity. Lack of physical exercise leads to weight gain, while lack of rest will lead to fatigue.

Systems and methods of embodiments of the present invention enable the user to monitor body signals and detect or predict timing most suitable for food consumption and physical activity. The system and method, according to some embodiments of the present invention also suggest serving size and serving content based on a previously programmed dietary plan, lifestyle data or input from the user.

In some embodiments of the present invention, the scheduling of daily activities based on the monitored physiological parameter is performed manually. The user reviews the logged physiological parameter data and based on data decides on the optimum time for meals, exercise and rest. The evaluation of biological rhythm can be done by “visual inspection” by a practitioner. The visual inspection includes presentation of the data in a chart that includes the raw signals, modeled signals, meal times and size, and physical activity. In other embodiments of the present invention the physiological parameter data is modeled and recommendations are reported to the user which indicate optimal times for meals, exercise and/or rest. Automatic evaluation of the biological rhythm includes comparison of the calculated parameters to a standard model or a model that is defined by a supervisor. The model for biological rhythm of specific physiological parameters is created from data collected from the user and specific model parameters are set depending on the person. Expected values for physiological parameters can be performed based also on demographic and clinical data of the user: particularly sex, age, weight, basal heart rate, VO₂ max etc. As biological rhythms vary, the physiological parameters of the model vary accordingly.

The optimal time for eating may be determined when the body is prepared in terms of gastrointestinal function. Alternatively the optimal time for eating may be determined by the biological rhythm of catabolic-anabolic cycle. In the context of the present invention, the optimal time for eating is indicated by specific physiological parameters which are monitored such as an increase of temperature of body skin and/or heart rate decrease. Other factors are optionally considered in addition including time from physical activity, time from the last meal, daily schedule of meals, and environmental temperature.

According to embodiments of the present invention, optimal time for exercise is determined when monitoring skin temperature and/or heart rate, after skin temperature peaks and starts to decrease, for instance two hours after eating or after resting. Alternatively, an exercise recommendation may be beneficial to induce a healthy biological rhythm. In this case, optimal exercise time may be indicated when heart rate or heat flow is relatively low for more than 3 hours. In this case, movement e.g. physical exercise is preferably performed in order to restore a healthy biological rhythm and initiate catabolic digestion of the last meal and before the next meal. High skin temperature with low heart rate is a marker for fatigue, and rest may be recommended

Additional or alternative typically parameters for monitoring according to different embodiments of the present invention include: heat flow, blood glucose, blood pressure, skin color, galvanic skin response (GSR) and movement (acceleration) that can be used for food scheduling. For example blood pressure is a good indicator of sympathetic-parasympathetic function, blood glucose is a good indicator of metabolism that can be used to identify when the body needs food, GSR can be used to identify sympathetic drive associated with effort or stress.

It should be noted that while the discussion herein is directed to scheduling of meals for the purpose of weight control, the principles of the present invention may be adapted for use in, and provide benefit for scheduling other activities such as physical exercise, school homework, mental activities, creative activities such as writing, musical composition and practicing as for musical performance. Further the monitoring mechanisms may be of any such mechanisms known in the art.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### EXAMPLE 1 OF BIOLOGICAL RHYTHM

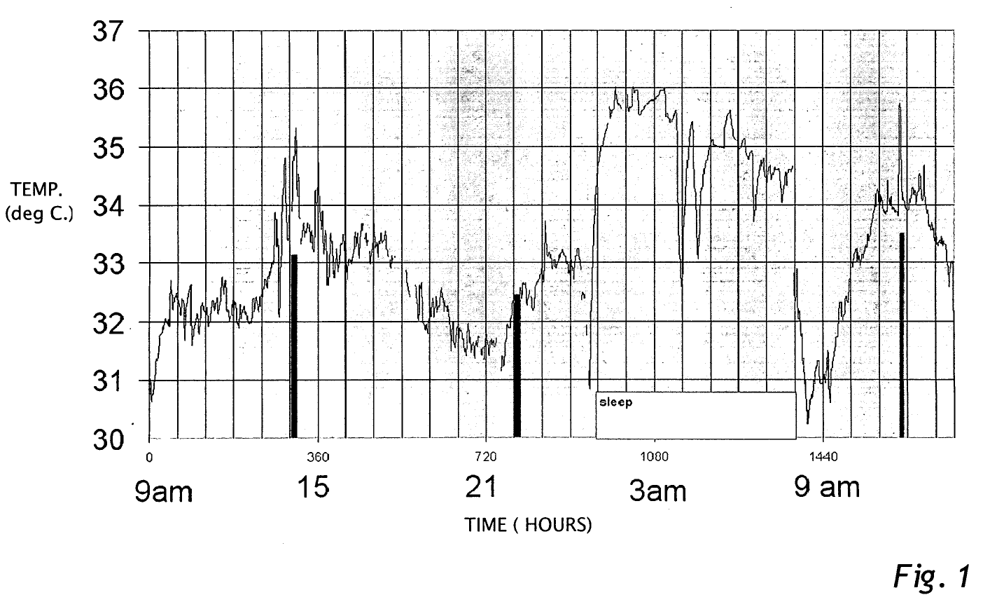

Referring now to the drawings, FIG. 1 illustrates a graph of skin temperature as a function of time in hours measured on the abdomen of a normal male subject. The skin temperature is measured with a standard thermistor and the results were plotted as a function of time. In FIG. 1, meal times are added and shown by vertical bars in which the height of the bars showing proportionally the size of the meal. Waking time at 8:00 may be inferred from the skin temperature data. From the period of time between awakening at 8:00 AM and approximately 10:00 AM the skin temperature rose to about 32 degrees on the average. The skin temperature remained essentially constant between 9:00 and 13:00 at 32 degrees C. Between 13:00 and 14:00 the skin temperature rose to about 33.5 degrees C. on the average. The period between 15:00 and 21:00 showed a decrease in skin temperature to 31.5 degrees C. The period between 21:00 and 24:00 showed an increase in skin temperature to about 32.5 degrees C. The subject went to sleep at about 01:00.

The biological rhythm of the subject is clear with a strong peak at the afternoon and a rise in the evening. The subject ate twice during the day while skin temperature is rising or near peak skin temperature. Sleep time shows characteristic high skin temperature (similar to inner body temperature) with oscillations perhaps characteristic to deep sleep stages. Our research has shown that most people with healthy biological rhythms report feeling of hunger and consequently eat during periods of skin temperature rise.

The data of FIG. 1 is consistent with a healthy biological rhythm during which skin temperature decreases, followed by rest during which skin temperature increases with eating preferably scheduled during rest periods while the skin temperature is increasing.

### System Description

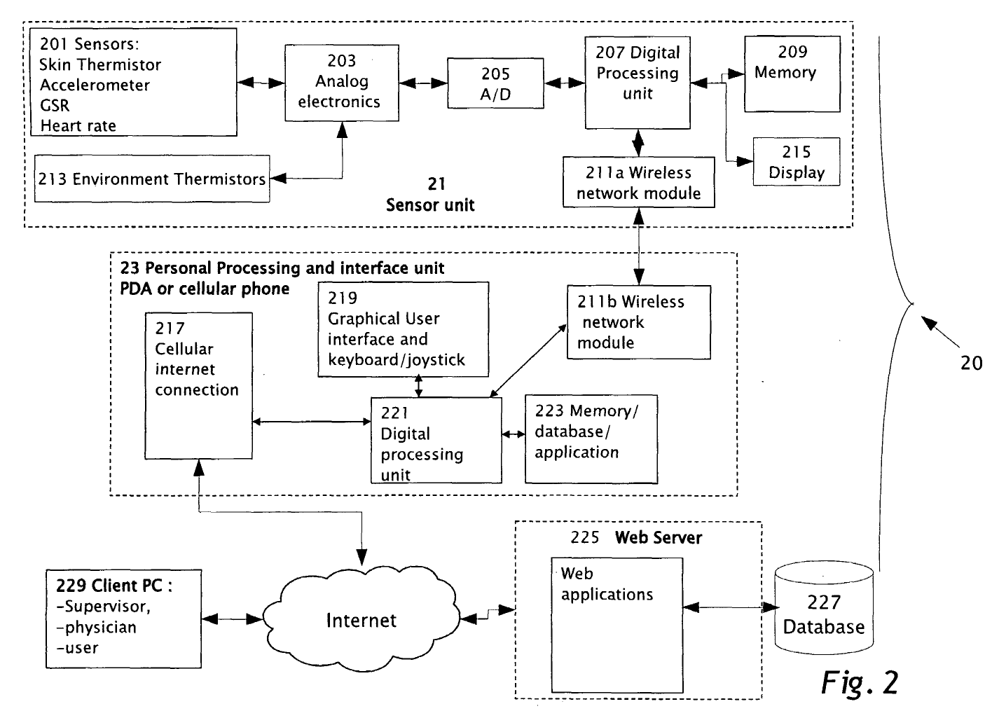

Reference is now made FIG. 2 which illustrates a system 20, according to embodiments of the present invention. System 20 includes a sensor unit 21 including one or more sensors 201 attached to the body. Sensor 201 that measures the one or more of following parameters:
Skin temperature on one or more places on the skin or body temperature is measured. Skin temperature sensor 201 is preferably a thermistor, commercial negative temperature coefficient (NTC) type 0.1 degrees Celsius accuracy. One or several thermistors are arranged in different positions on a belt or garment so that the thermistors contact skin at different points. In other embodiment of the present invention sensors 201 are placed on the arms, wrists, legs ankles to provide more comfort to the user. Sensor 201 is preferably thermally insulated from the external environment so that the temperature measured is minimally influenced by environmental temperature. Environmental temperature is optionally measured using one or several environmental thermistors 213 in order to normalize the results of skin or body temperature measurements, or identify and filter out the skin temperature fluctuation due to environment temperature sensor. Alternative embodiments include infrared sensitive elements such as pyroelectric sensors that enable surface temperature measurement without a direct contact with the skin. Heat flow may be estimated based on multiple temperature measurements on the body and in the environment of the body. Temperature differentials multiplied by respective heat capacities of objects, e.g bedding, in the environment are summed and compared with an approximate heat capacity of the body (or skin) multiplied by the temperature differential of the skin. Alternatively, or in addition heat flow is estimated as proportional to the temperature differential between the skin and the environment plus any additional heat losses due to air convection and evaporation of sweat.
heart rate—A heart rate monitor is a device which measures an electrocardiogram (ECG) preferably including dry electrodes and standard electronics that allows a user measure his/her heart rate and rhythm (R-R) in real time. The heart rate monitor usually includes two elements: a chest strap transmitter and a wrist receiver. The chest strap has electrodes in contact with the skin to monitor the electrical voltages in the heart. When a heartbeat is detected a radio signal is sent out which the receiver uses to determine the current heart rate. Heart rate variability (HRV) is a measure of variations in the heart rate. HRV is usually calculated by analyzing the time series of beat-to-beat intervals from an ECG sensor. Various measures of heart rate variability may subdivided into time domain, frequency domain and phase domain measures. A common frequency domain measure applies a discrete Fourier transform to the beat-to-beat interval time series. Several frequency bands of interest have been defined in humans. High Frequency band (HF) between 0.15 and 0.4 Hz. and low frequency band (LF) between 0.04 and 0.15 Hz.
Galvanic skin response (GSR) also known as electrodermal response (EDR), psychogalvanic reflex (PGR), or skin conductance response (SCR), is a known method of measuring the electrical resistance of the skin. GSR is conducted by attaching two leads to the skin, and acquiring a base measure. Then, as the activity being studied is performed, recordings are made from the leads. There are two ways to perform a GSR—in active GSR, current is passed through the body, with the resistance measured. In passive GSR, current generated by the body itself is measured.
Acceleration or body movement is measured in preferably in two or three dimensions. An accelerometer is an analog electronic device for calculating movement and position of the person wearing the accelerometer for calculation of body movement and body position in two or three directions. The accelerometer optionally measures also the direction of gravitation and thus body position may be monitored. Physical exercise is measured by averaging body acceleration after filtering noise including environmental noise. Algorithms known in the literature how to identify and filter out noise or remove artifacts from external factors such as from the acceleration of riding in a moving vehicle.
heat flow—is a measure of heat that is transferred from the body to the environment. The heat flow can be calculated based on skin temperature, environment temperature and perspiration (GSR or other sensors).

Sensor unit 21 typically includes analog electronics 203 connecting analog to digital converter A/D 205 which outputs a digital signal representing an output signal from sensor 201. The digital signal is input to a digital processing unit 207, typically a general purpose microprocessor an ASIC or other circuit. Digital processing unit 207 is connected to memory 209 in which the digital signal is logged and preferably a display unit 215. Digital processing unit 207 includes an interface, preferably a wireless interface 211, e.g. Bluetooth, connecting a personal processing unit 23. Personal Processing unit 23 includes an application installed in memory 223, the application performing a method for scheduling based on monitored physiological parameters, according to embodiments of the present invention. The term “processing unit” or “processor” are used herein interchangeably and refer to any computerized platform: a portable computer, a personal digital assistant (PDA) or a cellular telephone based on any processing technology such as a general microprocessor, ASIC or discrete digital and/or analog electronics. Personal processing unit 23 may include a digital processing unit 221, memory 223, a graphical user interface 219 and in the case of a cellular telephone, a cellular data (e.g. Internet) connection 217. Alternatively, sensor unit 21 and personal processing unit 23 may be packaged together as a single unit without requiring a cable or wireless interface 211. Personal processing unit 23 optionally uploads data to a Web server 225 attached to a Web accessible data base 227 and enables a user, or health care professional to access the data and schedule daily activities based on the data according to embodiments of the present invention.

In different embodiments of the present invention sensor unit 21 and/or personal processing unit 23 are integrated into a wrist watch with sensors 201 such as temperature monitor, heart rate monitor, and accelerometer or as a belt around the chest or abdomen with ECG electrodes, a thermistor and/or accelerometer.

User interface (UI) 219 is optionally used to add additional metadata for use in scheduling daily activities, in addition to the data provided by sensors 201. For instance, the user enters data such as hunger level, energy level, meal time, and size of meal. In addition UI 219 is used to present the scheduled activity to the user, by audiovisual signals that recommend a type of activity such as meal, light meal, and/or exercise. Alternatively, system 20 can send a message to the cellular phone or personal computer that will be integrated into the personal scheduler or diary. As the system may be distributed, having a wireless sensor connected to smart-phone 23 or a personal digital assistant (PDA) 23, UI 219 can be integrated into user interface of PDA 23, providing scheduling information.

Analysis of the biological rhythm of the user and scheduling of daily activities may be performed either in or shared by processing unit 207, processing unit 221, Web server 225 or client personal computer 229. Analysis and scheduling of activities may be can be performed in real time or not in real time subsequent to logging of data in memory 209 or memory 223 or subsequent to uploading of data and metadata to data base 227.

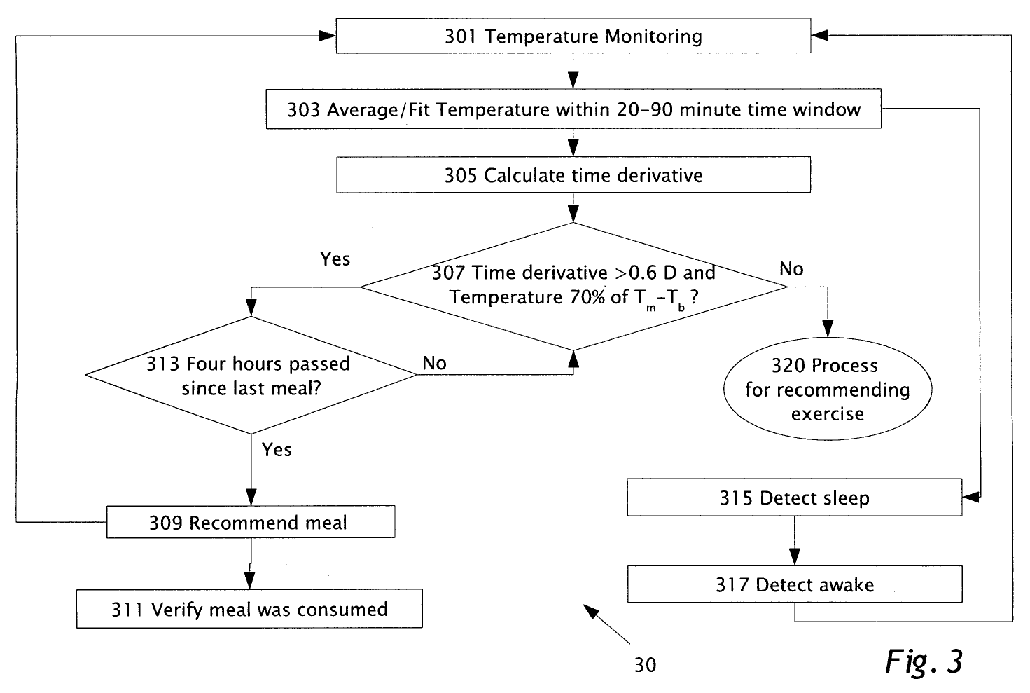

Reference is now made to FIG. 3, a flow diagram of a method 30 for scheduling meal times, according to an embodiment the present invention based on skin temperature monitoring. Method 30 establishes an individualized diet strategy, by recommending meal times, number of meals, and/or content of each meal.

Skin temperature monitoring (step 301) starts typically in the morning when the user wakes up and the skin temperature is measured. The skin temperature is averaged in time windows of typically 20-90 minutes and a base temperature T_{b} is established.
Alternatively, the measured temperature is fit to a polynomial of second or third order in a window of 20-90 minutes in order to smooth any high frequency fluctuations. A time derivative of the averaged temperature is calculated (step 305) from the base temperatures or from the fitted polynomial. In order to determine (step 309) recommended timing of an upcoming meal, the calculated time derivative is compared (decision block 307) with a previously determined value, e.g. 0.6*D, in which D is a characteristic temperature increase of for instance 2 degrees Celsius/hour. Alternatively or in addition the present temperature rise above base temperature T_{b} is compared with a characteristic temperature rise until the eating of meal. Tₘ−T_{b} is based on previously stored data. If the present temperature rise is for instance 70% of Tₘ−T_{b}, and a meal has not been recently eaten e.g. within four hours (decision block 313) then a meal is recommended within an hour. Preferably, the user is alerted in real time on display 215 that a meal is recommended (step 309). After recommending (step 309) a meal, the process continues with steps 301-305 and optionally in parallel, the program attempts to verify (step 311) that a meal was consumed based on the skin temperature data or system 20 receives metadata from the user regarding the timing and content of the meal consumed. Heart rate variability based on ECG can serve as an indicator for the quality and quantity of an ingested meal. After consumption of a meal is verified (step 311), temperature is typically monitored (step 301) however, recommendation of the next meal is postponed for certain period of time according to the diet strategy, which can be about four hours (step 313) as default. In parallel, system 20 detects sleep time (step 315), typically from a constant high skin temperature and detects (step 317) awake time based on a sudden drop in monitored skin temperature. After detection of awake time (step 317), the algorithm returns to the sub- process (steps 305-309) for recommending meal times.

Alternative embodiments include a finite state machine, where each state such as low skin temperature, high heart rate or physical activity, high skin temperature, low heart rate or low physical activity, increasing temperature from low to high, etc is defined by states. Rules for transition to a state of food or exercise recommendation is based on physiological parameters change and a model of healthy lifestyle including time between meals, exercise schedule etc.

The modeling of data and parameters such as D, Tₘ−T_{b} are preferably updated depending on menstrual period, seasons, geographical region, time-zone shifts due to air flight and/or working night shifts. Menstrual cycle influences the skin temperature that increases towards the end of the cycle and the variance decreases as well. The update of the model can be done by learning the statistical distribution of the skin temperature or other physiological parameters in the past 7-21 days.

A recommendation to rest is preferably issued when the skin temperature has reached high values that are typical to rest after meal and/or heart rate is below certain value. If in decision box 307, the skin temperature and/or heart rate are not behaving according to the previously stored model: for example the skin temperature is already high for many hours before an expected meal so that there is no possibility that the skin temperature will rise before the planned meal, then system 20 preferably recommends physical exercise (process 320) in order to activate sympathetic drive and burn the energy that was consumed before the next meal. If the user chooses not do physical exercise and the skin temperature stays high and no significant temperature variation occurs system 20 preferably recommends delaying the next meal and/or decreasing the size of the meal. By scheduling physical exercise between meals, delaying meals and reducing the size of the meals, a healthy biorhythm including variations in temperature and/or heart rate may be restored.

Energy snacks can be recommended before physical exercise if the last meal was more than 4 hours before the training.

Extreme external temperature in cold or hot conditions may influence the skin temperature results. In case of disease, body fever i.e. an average temperature rises above 37 degrees, the system reports that the user has a fever. Extreme skin temperature due to excessive exercise, dehydration, heat exhaustion and/or electrolyte depletion is reported to the user.

### EXAMPLE 2

### Healthy Biorhythm

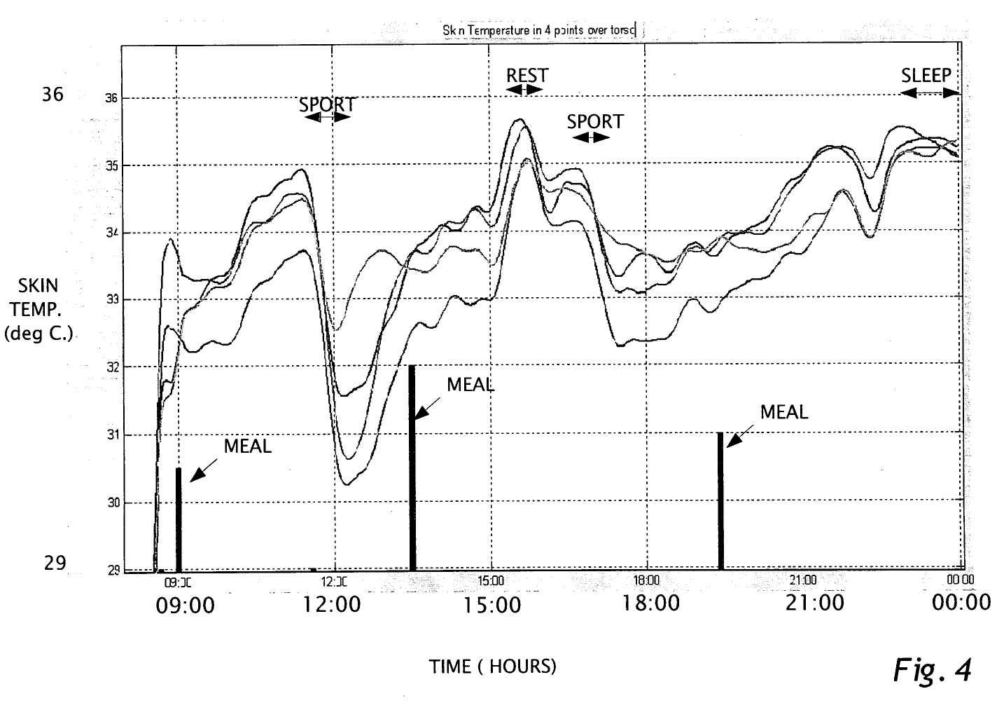

Reference is now made to FIG. 4, in which skin temperature measured at four points over the torso is graphed against time during 15 hours using system 20. The subject ate meals based on recommendations based on skin temperature using system 20 and 30. Sports rest and sleep are denoted by horizontal bars. Meals are denoted by vertical bars, the height of the bars is proportional to the meal size—breakfast, lunch, dinner. The graph of FIG. 4 is an example of healthy scheduling of exercise and meals relative to biological rhythm. The skin temperature and meal times are on the rise of the skin temperature after physical exercise and followed by rest. The amplitude of the skin temperature variation is quite large. As each day is different from the next biological rhythm monitoring, according to embodiments of the present invention enable adaptation to daily lifestyle changes.

### EXAMPLE 3

### Skin Temperature of Overweight Woman

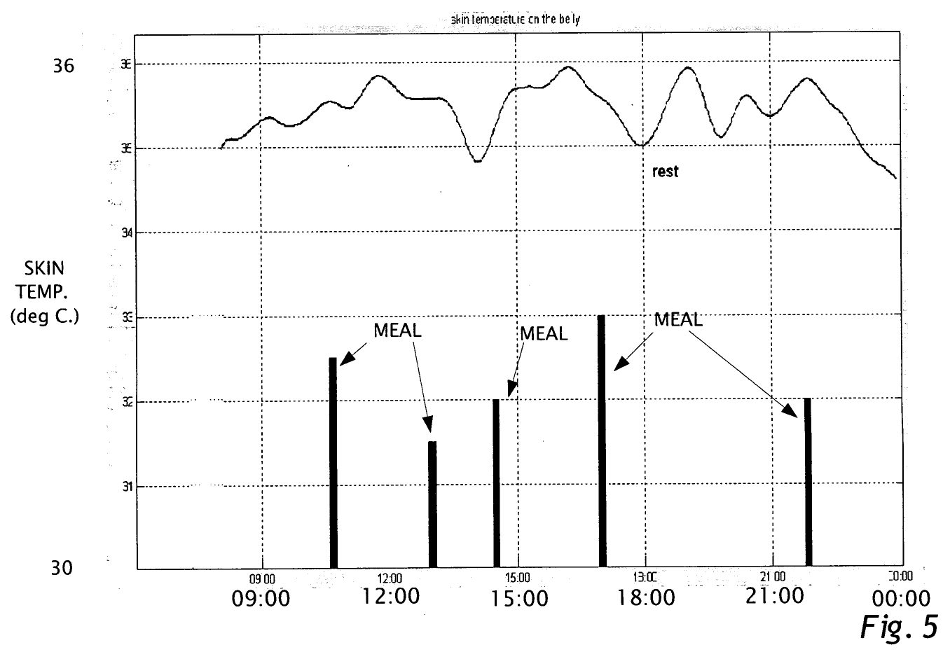

Reference is now made to FIG. 5, a graph of skin temperature measurements in the upper belly area and food intake of an overweight woman on a normal day. The subject is not on a prescribed diet and eats ad-libitum. The temperature variations are quite small between 35 and 36 degrees centigrade (36 degrees is almost the maximal limit of the skin temperature that is characteristic of sleep (as in the graphs of FIG. 1 and FIG. 4). The subject does not do physical exercise and has sedentary lifestyle. The food intake is quite frequent and meal times are weakly coordinated with the skin temperature rise. A weight management strategy might include working with the existing biological rhythm and advising to eat at the measured temperature rise and start doing small physical exercise at the times when skin temperature decreases. By enhancing the existing natural biological rhythm, the subject will be able to normalize her biological rhythm and more easily achieve weight control. Another approach is to drastically change the person's biological rhythm by the assigning scheduled meals and more rigorous exercise to establish a new biological rhythm. Food and exercise scheduling will train the biological rhythm to have variations as in the graph of FIG. 4. Once the biological rhythm is established the meals and exercise may be scheduled based on the skin temperature and/or on another monitored parameter using methods of the present invention similar to method 30.

### EXAMPLE 4

### Correlation of Skin Temperature with Heart Rate

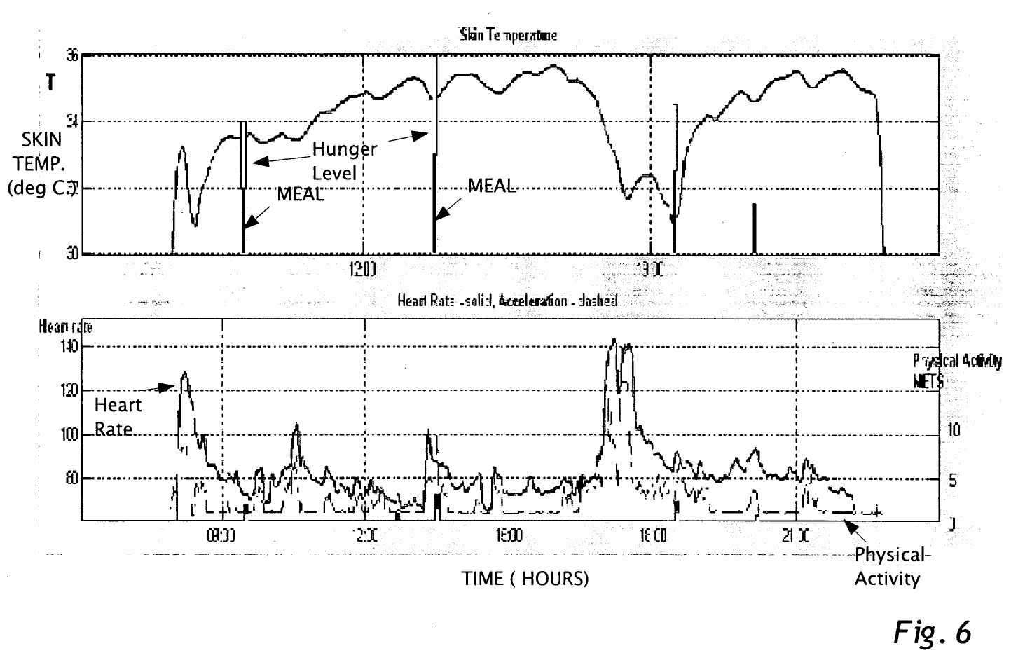

Reference is now made to FIG. 6 which illustrates the correlation between skin temperature, heart rate and physical activity. The upper graph depicts the skin temperature and the meal timing of a person who has not received any recommendations regarding meal time or exercise time scheduling. Dark vertical bars indicate meal times, the size of the meal is indicated by the length of the dark vertical bars while white vertical bars denote the degree of hunger. The lower graph shows heart rate in beats per minute, and physical activity of the same subject during same time. Physical activity is shown in metabolic equivalent (MET) level. Heart rate correlates proportionately with physical activity. Skin temperature is inversely correlated with heart rate and physical activity, with the variations in heart rate and physical activity leading the skin temperature change slightly. Body movement as measured by an accelerometer correlates with physical activity. It is therefore illustrated that skin temperature, heart rate, and physical activity can serve as predictors of hunger, optimal meal times and optimal exercise times, using different embodiments of the present invention.

Heart rate alone or in combination with acceleration and/or skin temperature and/or heat flow estimation based on temperature measurements can be used for scheduling the meals, exercise and rest. In the graph of FIG. 6, it can be seen that the heart rate decreases down to the basal heart rate when the person is hungry; and increases when the food is ingested. The heart rate increases during physical activity. Modeling of heart rate is performed for example by fitting heart rate to a polynomial in a time window of typically 20 to 90 minutes. The measured heart rate is compared to previously stored heart rate data and trends are identified of hunger and optimal scheduling of meal times. Correlation of heart rate measurements with simultaneous measurements of skin temperature and physical activity using an acceleration sensor can be beneficial for increasing the accuracy of optimal scheduling.

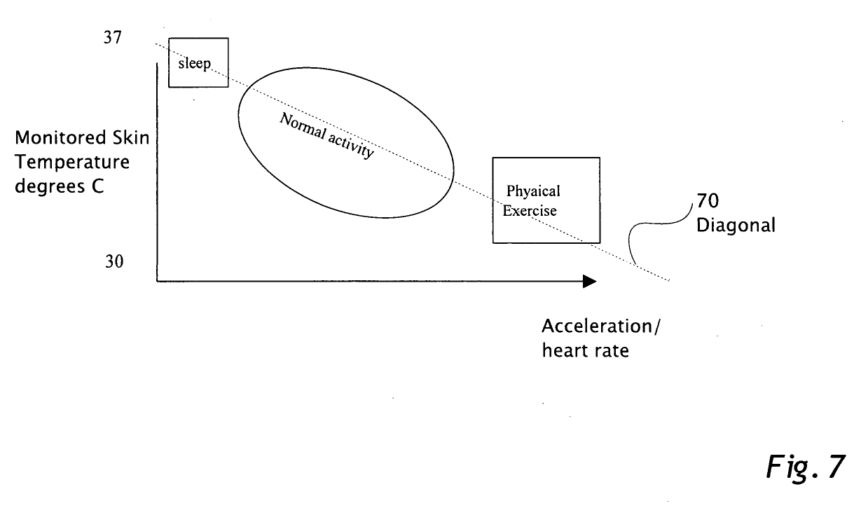

Reference is now made to FIG. 7 which illustrates a simplified “phase map” or finite state machine of human states as a function of skin temperature (ordinate in degrees Celsius) and either acceleration or heart rate (abscissa in arbitrary units. Diagonal 70 indicates normal conditions of sleep, daily activity and physical exercise. Since normal human conditions fall typically near diagonal 70, monitoring a single parameter e.g. skin temperature, heart rate, physical activity, heat flow is sufficient for accurate scheduling daily activities such as meals and exercise, according to embodiments of the present invention.

Evaluation of the biological rhythm may however include simultaneous processing of more than one of the monitored parameters and comparison to a standard model or another model that is defined by a supervisor. For example the evaluation of biological rhythm might include the following:
Variance of the skin temperature and/or heart rate between the meals.
Correlation between the meal and derivative of skin temperature and heart rate modeled signals.
Amount of physical activity in between the meals.
Correlation between the skin temperature and heart rate over the day.

Stress is not healthy and stress is known to adversely affect cardiovascular health. Eating while in stress is also not healthy. System 20 may be configured to identify stress as indicated for example by heart rate variability in combination with low skin temperature in combination with low activity (which is abnormal). System 20 preferably recommends subjects to do stress releasing exercise or treatments e.g. ingest medications in order to relieve stress. While in a state of stress, food will preferably not be recommended, but rather after the skin temperature rises and stress is relieved.

Sleep balance in obese subjects is important as they suffer from imbalanced circadian rhythm and nocturnal eating. System 20 may be programmed to identify the situation when the subject has disturbances in circadian rhythm and might have difficulty falling asleep. A sleep inducing drug is optionally recommended by system 20. Natural preparation to sleep is indicated by a rise in the skin temperature at the late evening and a decreasing heart rate which prepares the body for diurnal sleep. If the skin temperature goes down or heart rate does not go down (stays high) in the late evening, then there is an indication that the subject has disturbances in circadian rhythm and might difficulty falling asleep. In this case the system may recommend taking a sleep inducing drug.

While the invention has been described with respect to a limited number of embodiments, it will be appreciated that many variations, modifications and other applications of the invention may be made. Particularly adaptive and learning algorithm that identify physiological signs change that is characteristic to the food intake and exercise.

1. A computerized system for scheduling at least one daily activity of a user, the computerized system comprising:
(a) at least one sensor adapted for attaching to the body of the user, said at least one sensor adapted for monitoring at least one physiological parameter of the body thereby producing physiological data representative of said at least one physiological parameter during a time period; and
(b) a processing unit operatively attachable to memory, wherein the processing unit is programmed for the scheduling of the at least one daily activity based on the physiological data and on at least one previously stored value.

2. The computerized system according to claim 1, wherein the at least one daily activity is eating of a meal, exercise or rest of the user.

3. The computerized system according to claim 1, wherein said at least one previously stored value is updated using statistical analysis of said physiological data relating to biological rhythm of the body.

4. The computerized system according to claim 1, wherein the at least one physiological parameter is skin temperature, heat flow from the body, heart rate, heart rate variability, galvanic skin response, blood pressure, blood glucose, skin color, accelerometer, body movement and physical activity.

5. The computerized system according to claim 1, further comprising:
(c) an input mechanism for entering by the user metadata pertaining to the user, said metadata selected from the group consisting of: a hunger level, an energy level, a meal time, and a size of a meal,
wherein said scheduling is further based on the metadata.

6. The computerized system according to claim 4, wherein the at least one daily activity is an eating of a meal, and wherein the processing unit is programmed to recommend to the user to eat said meal during a portion of the time period when a characteristic change occurs of said physiological parameter.

7. The computerized system according to claim 1, wherein said physiological data is indicative of a high stress level, and wherein said scheduling includes delaying a meal until said stress level is reduced.

8. The computerized system according to claim 1, wherein the at least one physiological parameter is heat flow, heart rate variability, galvanic skin response, blood pressure, blood glucose, skin color, body movement and physical activity.

9. A method for scheduling at least one daily activity of a user, the method comprising:
(a) providing at least one sensor attachable to the body of the user, said at least one sensor monitoring at least one physiological parameter of the body thereby producing physiological data representative of said at least one physiological parameter during a time period; and
(b) scheduling of the at least one daily activity based on the physiological data and on at least one previously stored value.

10. The method according to claim 9, further comprising:
(c) updating said at least one previously stored value using statistical analysis of said physiological data.

11. The method according to claim 9, wherein said at least one daily activity is eating of a meal, exercise or rest of the user.

12. The method according to claim 9, wherein said at least one physiological parameter is skin temperature or heart rate.

13. The method according to claim 12, wherein the at least one daily activity is exercise, further comprising:
(c) programming the processing unit to recommend to the user to exercise when at least one condition occurs said at least one condition selected from the group consisting of: after said skin temperature peaks and begins to decrease and after said heart rate reaches a minimum and begins to increase.

14. The method according to claim 12, wherein the at least one daily activity is rest, further comprising:
(c) programming the processing unit to recommend to the user to rest when at least one condition occurs said at least one condition is: the skin temperature has reached a value higher than a previously stored threshold skin temperature value and the heart rate is below a previously stored threshold heart rate value.

15. The method according to claim 12, wherein during said time period when at least one condition occurs then said scheduling includes postponing a meal and recommending exercise, said at least one condition selected from the group consisting of: said skin temperature is above a previously defined threshold for longer than a previously defined time interval and said heart rate is lower than a previously defined threshold for longer than a previously defined time interval.

16. The method according to claim 12, wherein the at least one daily activity is eating of a meal, further comprising:
(c) programming the processing unit to recommend to the user to eat said meal during a portion of the time period when at least one condition occurs said at least one condition selected from the group consisting of said skin temperature is rising and said heart rate is falling.

17. The method according to claim 9, further comprising:
(c) entering by the user metadata pertaining to the user, said metadata selected from the group consisting of hunger level, energy level, meal time, and size of meal; wherein said scheduling is further based on the metadata.

18. The method according to claim 9, wherein said physiological data is indicative of a high stress level, wherein said scheduling includes delaying a meal until said stress level is reduced.

19. The method according to claim 9, wherein the at least one physiological parameter is galvanic skin response, blood pressure, blood glucose, skin color, body movement and physical activity.

20. The method according to claim 9, wherein the scheduling is based on the physiological data of a single said at least one physiological parameter.

21. A computer readable medium encoded with processing instructions for causing a processor to execute a method for collecting physiological data from at least one sensor attachable to the body of the user, said at least one sensor monitoring at least one physiological parameter of the body thereby producing said physiological data representative of said at least one physiological parameter during a time period and scheduling of at least one daily activity based on the physiological data and based on at least one previously stored value.
